(19) United States
(12) Patent Application Publication
(10) Pub. No.: US 2019/0117681 A1
(43) Pub. Date: Apr. 25, 2019

(54) GLYCOGEN AND PHYTOGLYCOGEN NANOPARTICLES AS IMMUNOSUPPRESSIVE COMPOUNDS, AND COMPOSITIONS AND METHODS OF USE THEREOF

(71) Applicant: MIREXUS BIOTECHNOLOGIES INC., Guelph (CA)

(72) Inventors: Anton KORENEVSKI, Guelph (CA); Stephanie DEWITTE-ORR, Waterloo (CA); Katelin SPITERI, Waterloo (CA); Lisa Suzanne BERTOLO, Guelph (CA); Sheetij GHOSHAL, Mississauga (CA)

(21) Appl. No.: 16/098,715

(22) PCT Filed: May 4, 2017
(86) PCT No.: PCT/CA2017/050545

§ 371 Date: Nov. 2, 2018

Related U.S. Application Data
(60) Provisional application No. 62/454,424, filed on Feb. 3, 2017.
(60) Provisional application No. 62/331,662, filed on May 4, 2016.

(51) Int. Cl. A61K 31/715; A61K 47/69; A61K 9/00; C12N 5/00; A61K 9/51; A61P 31/14

(52) U.S. Cl. CPC A61K 31/715; A61K 47/6939; A61K 9/0014; A61K 48/00; A61K 9/51; A61P 31/14; C12N 5/0018

(57) ABSTRACT

Compounds and compositions comprising glycogen or phytoglycogen nanoparticles are provided that suppress type I interferon innate immune responses. The glycogen or phytoglycogen nanoparticles in the composition are suitably cationized, in one embodiment, functionalized with an amino group.

This application claims priority from U.S. Applications No. 62/331,662 and No. 62/454,424, which are incorporated herein by reference.

### TECHNICAL FIELD

This application relates to immunomodulator compositions.

### BACKGROUND OF THE ART

Glycogen is a short-term energy storage material in animals. In mammals, glycogen occurs in muscle and liver tissues. It is comprised of 1,4-glucan chains, highly branched via α-1,6-glucosidic linkages with a molecular weight of 10⁶-10⁸ Daltons. Glycogen is present in animal tissues in the form of dense particles with diameters of 20-200 nm. Glycogen is also found to accumulate in microorganisms, e.g., in bacteria and yeasts.

Phytoglycogen is a polysaccharide that is very similar to glycogen, both in terms of its structure and physical properties. It is distinguished from glycogen based on its plant-based sources of origin. The most prominent sources of phytoglycogen are kernels of sweet corn, as well as specific varieties of rice, barley, and sorghum.

### BRIEF SUMMARY

In one embodiment, there is provided a use of an effective amount of glycogen or phytoglycogen nanoparticles for suppressing an anti-viral response in a cell, a cell culture, a tissue, or a subject. Also provided is a method of suppressing an anti-viral response in a cell, a cell culture, a tissue, or a subject comprising introducing or administering an effective amount of glycogen or phytoglycogen nanoparticles to the cell, the cell culture, the tissue, or the subject.

In one embodiment, the glycogen or phytoglycogen nanoparticles are cationized.

In one embodiment, the glycogen or phytoglycogen nanoparticles are amine-modified.

In one embodiment, the glycogen or phytoglycogen nanoparticles are modified with a short-chain quaternary ammonium compound comprising at least one alkyl moiety having from 1 to 16 carbon atoms, unsubstituted or substituted with one or more N, O, S, or halogen atoms.

In one embodiment, the nanoparticles have an average particle diameter of between about 30 nm and about 150 nm. In one embodiment, at least 90% or substantially all the nanoparticles have an average diameter of between about 40 nm and about 140 nm, about 50 nm and about 130 nm, about 60 nm and about 120 nm, about 70 nm and about 110 nm, about 80 nm and about 100 nm, about 30 nm and about 40 nm, about 40 nm and about 50 nm, about 50 nm and about 60 nm, about 60 nm and about 70 nm, about 70 nm and about 80 nm, about 80 nm and about 90 nm, about 90 nm and about 100 nm, about 100 nm and about 110 nm, about 110 nm and about 120 nm, about 120 nm and about 130 nm, about 130 nm and about 140 nm, or about 140 nm and about 150 nm.

In one embodiment, the nanoparticles are not further conjugated to another molecule.

In another embodiment, the nanoparticles are further conjugated to one or more small molecules, wherein the molecule is a hydrophilicity modifier, pharmokinetic modifier, a biologically active modifier or a detectable modifier.

In one embodiment, the subject is a viral-vector based gene therapy patient.

The use and methods as described herein may be for the treatment or prevention of a disease or condition.

In one embodiment, the disease or condition is an autoimmune disease.

In one embodiment, the disease or condition is an inflammatory disease.

In one embodiment, the disease or condition is pain.

In one embodiment, the disease or condition is sepsis.

In one embodiment, the composition is for topical administration.

In one embodiment, the composition is for systemic administration.

The use and methods as described herein may be for the manufacture of vaccines.

In one embodiment, the use is for enhancing viral growth and replication in an infected cell culture in the manufacture of vaccines.

Also provided is a combination therapy comprising a viral-vector based gene therapy and glycogen or phytoglycogen nanoparticles. In one embodiment, the glycogen or phytoglycogen nanoparticles are cationized. In one embodiment, the glycogen or phytoglycogen nanoparticles are amine-modified. In one embodiment, the glycogen or phytoglycogen nanoparticles are modified with a short-chain quaternary ammonium compound comprising at least one alkyl moiety having from 1 to 16 carbon atoms, unsubstituted or substituted with one or more N, O, S, or halogen atoms.

In one embodiment, the viral-vector based gene therapy is an adenovirus.

### BRIEF DESCRIPTION OF THE DRAWINGS

FIG. 1 is a schematic drawing of a phytoglycogen/glycogen nanoparticle.

FIG. 2 shows the influence of phytoglycogen (PHX) and chemically modified phytoglycogen on rainbow trout gonadal cells (RTG-2) cell line viability. AB—Alamar Blue, CFDA—Carboxyfluorescein Diacetate, Acetoxymethyl Ester. PHX—phytoglycogen OSA3—OSA-phytoglycogen, DS=0.02 NH2—Amino-phytoglycogen, DS=0.1 Q151—N-(2-hydroxy)propyl-3-trimethyl ammonium-phytoglycogen, DS=1.00

FIG. 3 shows changes in MX1 (a representative innate immune gene) transcript expression in RTgill W-1 cells after 72 hours or 144 hours infection with VHSV-IVb in the presence or absence of PHX-NH2. RTgill W-1 cells were treated with media alone (control), VHSV-IVb and VHSV-IVb with 0.5 ng/mL or 0.05 ng/mL of PHX-NH2 for 72 or 144 hours. MX1 transcript levels were measured using qRT-PCR.

FIG. 4 shows changes in Mx1 transcript expression in RTG-2 cells after a 24 h infection with chum salmon reovirus (CSV) (CSV; TCID50=1.995×104) in the presence or absence of PHX-NH₂. RTG2 cells were treated with media alone (control), CSV and CSV with 0.5 ng/mL of PHX-NH₂ for 24 hours. Twenty four hours post-infection transcript levels of, Mx1, were measured using qRTPCR. Expression was calculated using the ΔΔCq method, normalized to β-actin and relative to CSV infected cells. Data was analyzed using a one-way ANOVA with a Tukey's post-test, *** P≤0.001.

FIG. 5 shows cytopathic effect of CHSE-214 cells after a 72 hour infection with CSV in the presence or absence of unmodified PHX or PHX-NH2. CHSE-214 cells were treated with CSV, CSV with 0.05 ng/mL of unmodified PHX, or CSV with 0.05 ng/mL PHX-NH2 for 72 hours. CHSE-214 cells were then fixed, stained and area covered by syncytia/total area (=% syncytia) was quantified.

FIG. 6 shows internalization of Cy5.5-labelled PHX particles by RTG-2 cells. Fluorescence confocal images of RTG-2 cells incubated with Cy5.5-Phytoglycogen nanoparticles. RTG-cells were treated for 2 hours or 20 hours in PBS alone (untreated control cells) or cells treated with PHX-CY5.

FIG. 7 shows quantification of fluorescent signals in organs imaged ex vivo at 30 min and 24 h after i.v. injection in naïve nude CD-1 mice. The average fluorescence concentration data, suggests that in addition to the liver and kidney, high signal can also be detected in lung and heart. The fluorescence concentrations at 30 mins are higher than at 24 hrs. Pre-scan data indicates the fluorescence concentration data for a mouse not injected with Cy5.5-Phytoglycogen (i.e. background autofluorescence). Data are presented as mean+/−SD.

FIG. 8 shows quantification of fluorescent signals in brain imaged ex vivo at 30 min and 24 h after i.v. injection of Cy5.5-Phytoglycogen in naïve nude CD-1 mice. The data indicate that compared to pre-scan (autofluorescence level), there are measureable signals in the brain from Cy5.5-Phytoglycogen. The signal is highest at 30 mins and goes down slowly over time at 24 hrs.

FIG. 9 shows quantification of PHX-NH2 effects on CSV production in CHSE-214 cells, using rocking method. PHX-NH2 caused a 943.96% increase in CSV titre (SEM=43.97) over CSV alone. (p>0.05).

FIG. 10 shows quantification of PHX-NH2 effects on VHSV-IVb production in epithelioma papulosum cyprinid (EPC) cells, using two treatments, rocking and pre-treatment methods. With the pretreatment method, PHX-NH2 caused a 147.96% increase in VHSV-IVb titre (SEM=125.2) over VHSV-IVb alone. With the rocking method, PHX-NH2 decreased VHSV-IVb titre by 26% (SEM=25.88%). (p<0.05).

FIG. 11 shows quantification of PHX-NH2 effects on PHX-NH2 effects on infectious pancreatic necrosis virus (IPNV) production in CHSE-214 cells, using pretreatment method. PHX-NH2 caused a 4.72% increase in IPNV titre (SEM=1.99) over IPNV alone. (p=0.62).

### DETAILED DESCRIPTION

As used herein “immunotherapy” refers to treating or preventing disease by inducing, enhancing or suppressing an immune response. An immunotherapy designed to elicit or amplify an immune response may be referred to as an activation immunotherapy, while an immunotherapy designed to reduce or suppress is referred to as a suppression immunotherapy. An immunomodulator is used herein to refer to an active agent used in immunotherapy. An immunosuppressive refers to an immunomodulator used in suppression immunotherapy.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

As used herein “subject” refers to an animal being given immunotherapy, in one embodiment a mammal, in one embodiment a human patient. As used herein “treatment” and grammatical variations thereof refers to administering a compound or composition of the present invention in order to suppress an immune response. This treatment may be to effect an alteration or improvement of a disease or condition, which may include alleviating one or more symptoms thereof, or the use may be prophylactic to prevent a disease or condition. The treatment may require administration of multiple doses at regular intervals or prior to onset of the disease or condition to alter the course of the disease or condition

The present disclosure relates to glycogen or phytoglycogen nanoparticles for use in immunosuppresion. For example, the nanoparticles may be used in organ transplant.

The present disclosure relates to pharmaceutical and biomedical compositions comprising glycogen or phytoglycogen nanoparticles for use in immunotherapy.

The present disclosure relates to glycogen or phytoglycogen nanoparticles for use in the manufacture of vaccines.

The present disclosure relates to combination therapy comprising a viral-vector based gene therapy and glycogen or phytoglycogen nanoparticles.

In one embodiment, there is provided compounds and compositions capable of suppressing type I interferon responses in vertebrates. As innate immune responses are highly conserved between vertebrates, the findings of the Examples can be extended to other animals, including humans. The IFN-suppresive capabilities of the compounds and compositions described herein have a number of potential therapeutic uses, including, without being limited to, treatment of septic shock and in combination with adenovirus for gene therapy.

Phytoglycogen is composed of molecules of α-D glucose chains having an average chain length of 11-12, with 1→4 linkage and branching point occurring at 1→6 and with a branching degree of about 6% to about 13%. In one embodiment, phytoglycogen includes both phytoglycogen derived from natural sources and synthetic phytoglycogen. As used herein the term “synthetic phytoglycogen” includes glycogen-like products prepared using enzymatic processes on substrates that include plant-derived material e.g. starch.

The yields of most known methods for producing glycogen or phytoglycogen and most commercial sources are highly polydisperse products that include both glycogen or phytoglycogen particles, as well as other products and degradation products of glycogen or phytoglycogen.

In one embodiment, monodisperse glycogen or phytoglycogen nanoparticles are used. In a preferred embodiment, phytoglycogen nanoparticles are used. In a further preferred embodiment, monodisperse phytoglycogen nanoparticles are used. In one embodiment, the monodisperse phytoglycogen nanoparticles are PhytoSpherix™ produced by Mirexus Biotechnologies, Inc.

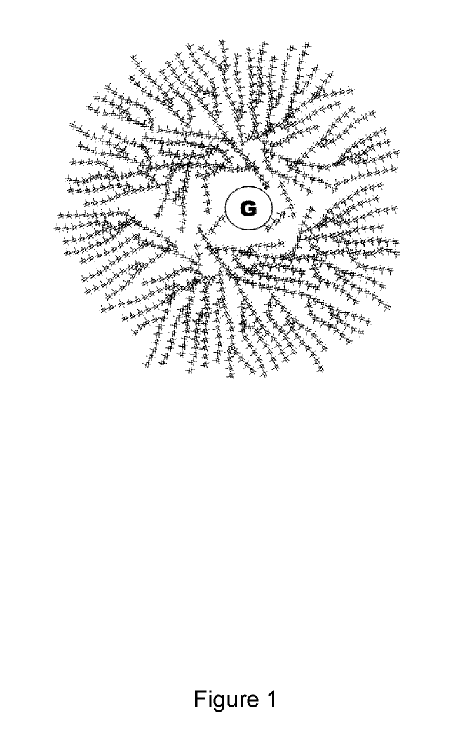

As shown in FIG. 1, each phytoglycogen/glycogen particle is a single molecule, made of highly-branched glucose homopolymer characterized by very high molecular weight (up to 10⁷ Da). Spherical and can be manufactured with different sizes, in the range of 30 to 150 nm in diameter by varying the starting material and filtering steps. The high density of surface groups on the phytoglycogen/glycogen particles results in a variety of unique properties of phytoglycogen/glycogen nanoparticles, such as fast dissolution in water, low viscosity and shear thinning effects for aqueous solutions at high concentrations of phytoglycogen/glycogen nanoparticles. This is in contrast to high viscosity and poor solubility of linear and low-branched polysaccharides of comparable molecular weight. Furthermore, it allows formulation of highly concentrated (up to 30%) stable dispersions in e.g. water or DMSO.

In one embodiment, phytoglycogen refers to monodisperse phytoglycogen nanoparticles manufactured according to methods described herein. The described methods enable production of substantially spherical nanoparticles, each of which is a single phytoglycogen molecule.

The nanoparticles as taught herein have a number of properties that make them particularly suitable for use in immunosuppressive pharmaceutical compositions.

These phytoglycogen nanoparticles are non-toxic, have no known allergenicity, and can be degraded by glycogenolytic enzymes (e.g. amylases and phosphorylases) of the human body. The products of enzymatic degradation are non-toxic molecules of glucose.

Glycogen and phytoglycogen nanoparticles are generally photostable and stable over a wide range of pH, electrolytes, e.g. salt concentrations.

Further, many existing drugs are rapidly eliminated from the body leading to a need for increased dosages. The compact spherical nature of phytoglycogen and glycogen nanoparticles is associated with efficient cell uptake, while the highly branched nature of glycogen and phytoglycogen is associated with slow enzymatic degradation. Further, the high molecular weight (10⁶-10⁷ Da) is believed to be associated with longer intravascular retention time.

Glycogen and phytoglycogen nanoparticles may have particular utility in compositions directed to diabetics based on a slower in vivo rate of digestion as compared to starch.

Phytoglycogen/glycogen nanoparticles have properties that address a number of requirements for materials used in pharmaceutical and biomedical applications: predictable biodistribution in different tissues and associated pharmokinetics; hydrophilicity; biodegradability; and non-toxicity.

As demonstrated in the Examples, the present inventors have found that immunosuppressive compounds as provided herein can be accumulated intracellularly by different types of cells.

United States patent application publication no. United States 20100272639 A1, assigned to the owner of the present application and the disclosure of which is incorporated by reference in its entirety, provides a process for the production of glycogen nanoparticles from bacterial and shell fish biomass. The processes disclosed generally include the steps of mechanical cell disintegration, or by chemical treatment; separation of insoluble cell components by centrifugation; elimination of proteins and nucleic acids from cell lyzate by enzymatic treatment followed by dialysis which produces an extract containing crude polysaccharides, lipids, and lypopolysaccharides (LPS) or, alternatively, phenol-water extraction; elimination of LPS by weak acid hydrolysis, or by treatment with salts of multivalent cations, which results in the precipitation of insoluble LPS products; and purification of the glycogen enriched fraction by ultrafiltration and/or size exclusion chromatography; and precipitation of glycogen with a suitable organic solvent or a concentrated glycogen solution can be obtained by ultrafiltration or by ultracentrifugation; and freeze drying to produce a powder of glycogen. Glycogen nanoparticles produced from bacterial biomass was characterized by MWt 5.3-12.7×106 Da, had particle size 35-40 nm in diameter and was monodisperse.

Methods of producing monodisperse compositions of phytoglycogen are described in the International patent application entitled “Phytoglycogen Nanoparticles and Methods of Manufacture Thereof”, published under the international application publication no. WO2014/172786, assigned to the owner of the present application, and the disclosure of which is incorporated by reference in its entirety. In one embodiment, the described methods of producing monodisperse phytoglycogen nanoparticles include: a. immersing disintegrated phytoglycogen-containing plant material in water at a temperature between about 0 and about 50° C.; b. subjecting the product of step (a.) to a solid-liquid separation to obtain an aqueous extract; c. passing the aqueous extract of step (b.) through a microfiltration material having a maximum average pore size of between about 0.05 μm and about 0.15 μm; and d. subjecting the filtrate from step c. to ultrafiltration to remove impurities having a molecular weight of less than about 300 kDa, in one embodiment, less than about 500 kDa, to obtain an aqueous composition comprising monodisperse phytoglycogen nanoparticles. In one embodiment of the method, the phytoglycogen-containing plant material is a cereal selected from corn, rice, barley, sorghum or a mixture thereof. In one embodiment, step c. comprises passing the aqueous extract of step (b.) through (c.1) a first microfiltration material having a maximum average pore size between about 10 μm and about 40 μm; (c.2) a second microfiltration material having a maximum average pore size between about 0.5 μm and about 2.0 μm, and (c.3) a third microfiltration material having a maximum average pore size between about 0.05 and 0.15 μm. The method can further include a step (e.) of subjecting the aqueous composition comprising monodisperse phytoglycogen nanoparticles to enzymatic treatment using amylosucrose, glycosyltransferase, branching enzymes or any combination thereof. The method avoids the use of chemical, enzymatic or thermo treatments that degrade the phytoglycogen material. The aqueous composition can further be dried.

In one embodiment, the composition is obtained from sweet corn (Zea mays var. saccharata and Zea mays var. rugosa). In one embodiment, the sweet corn is of standard (su) type or sugary enhanced (se) type. In one embodiment, the composition is obtained from dent stage or milk stage kernels of sweet corn. Unlike glycogen from animal or bacterial sources, use of phytoglycogen reduces the risk of contamination with prions or endotoxins, which could be associated with these other sources.

The methods of producing phytoglycogen nanoparticles as detailed in Example 1 and in the international patent application entitled “Phytoglycogen Nanoparticles and Methods of Manufacture Thereof”, are amenable to preparation under pharmaceutical grade conditions.

The polydispersity index (PDI) of a composition of nanoparticles can be determined by the dynamic light scattering (DLS) technique and, in this embodiment, PDI is determined as the square of the ratio of standard deviation to mean diameter (PDI=(σ/d)2. PDI can also be expressed through the distribution of the molecular weight of polymer and, in this embodiment, is defined as the ration of Mw to Mn, where Mw is the weight-average molar mass and Mn is the number-average molar mass (hereafter this PDI measurement is referred to as PDI*). In the first case, a monodisperse material would have a PDI of zero (0.0) and in the second case the PDI* would be 1.0.

In one embodiment, there is provided a pharmaceutical composition that comprises, consists essentially of, or consists of a composition of monodisperse glycogen or phytoglycogen nanoparticles. Suitably, the nanoparticles are functionalized and, in particular, cationized, as discussed further below. In one embodiment, the pharmaceutical composition comprises, consists essentially of, or consists of a composition of monodisperse glycogen or phytoglycogen nanoparticles having a PDI of less than about 0.3, less than about 0.2, less than about 0.15, less than about 0.10, or less than 0.05 as measured by dynamic light scattering. In one embodiment, the pharmaceutical composition comprises, consists essentially of, or consists of a composition of monodisperse glycogen or phytoglycogen nanoparticles having a PDI* of less than about 1.3, less than about 1.2, less than about 1.15, less than about 1.10, or less than 1.05 as measured by SEC MALS.

In one embodiment, the pharmaceutical composition comprises, consists essentially of, or consists of a composition of monodisperse glycogen or phytoglycogen nanoparticles having an average particle diameter of between about 30 nm and about 150 nm. In one embodiment, the pharmaceutical composition comprises, consists essentially of, or consists of a composition of monodisperse glycogen or phytoglycogen nanoparticles having an average particle diameter of about 60 nm to about 110 nm. In other embodiments, there is provided compositions comprising, consisting essentially of, or consisting of, nanoparticles having an average particle diameter of about 40 to about 140 nm, about 50 nm to about 130 nm, about 60 nm to about 120 nm, about 70 nm to about 110 nm, about 80 nm to about 100 nm.

### Chemical Modification of Phytoglycogen and Glycogen Nanoparticles

To impart specific properties to glycogen and phytoglycogen nanoparticles, they can be chemically modified via numerous methods common for carbohydrate chemistry.

Accordingly, in one embodiment, the nanoparticles are modified. The resulting products are referred to herein interchangeably as functionalized or modified nanoparticles or derivatives. Functionalization can be carried out on the surface of the nanoparticle, or on both the surface and the interior of the particle, but the structure of the glycogen or phytoglycogen molecule as a single branched homopolymer is maintained. In one embodiment, the functionalization is carried out on the surface of the nanoparticle. As will be understood by those of skill in the art, chemical modifications should be non-toxic and generally safe for human consumption.

In some embodiments of the present invention, it is advantageous to change the chemical character of glycogen/phytoglycogen from its hydrophilic, slightly negatively charged native state to be positively charged, or to be partially or highly hydrophobic. J. F Robyt, Essentials of Carbohydrate Chemistry, Springer, 1998; and M. Smith, and J. March, March's Advanced Organic Chemistry: Reactions, Mechanisms, and Structure Advanced Organic Chemistry, Wiley, 2007 provides certain examples of chemical processing of polysaccharides.

The nanoparticles can be either directly functionalized or indirectly, where one or more intermediate linkers or spacers can be used. The nanoparticles can be subjected to one or more than one functionalization steps e.g. two or more.

Various derivatives can be produced by chemical functionalization of hydroxyl groups of glycogen/phytoglycogen. Such functional groups include, but are not limited to, nucleophilic and electrophilic groups, and acidic and basic groups, e.g., carbonyl groups, amine groups, thiol groups, carboxylic groups, and hydrocarbyl groups such as alkyl, vinyl and allyl groups.

In one embodiment, the glycogen or phytoglycogen nanoparticles are modified to have at least a positive surface charge (cationized). In one embodiment, the nanoparticles are modified by an amine (NH₂) group.

Suitably, glycogen or phytoglycogen nanoparticles are modified with amino groups, which can be primary, secondary, tertiary, or quaternary amino groups.

In one embodiment, the functionalized nanoparticles are modified with a short-chain quaternary ammonium compound. The short-chain quaternary ammonium compound includes at least one alkyl moiety having from 1 to 16 carbon atoms, unsubstituted or substituted with one or more N, O, S, or halogen atoms.

In certain embodiments, two or more different chemical compounds are used to produce multifunctional derivatives.

The reactivity of primary hydroxyl groups on glucose subunits (in aqueous environment) is low. Even so, reactions are possible with epoxides, anhydrides or alkyl halides forming the corresponding ether or ester linkages. Water-soluble chemicals with epoxide or anhydride functionalities react at basic pH (e.g. 8-11) with glycogen and phytoglycogen nanoparticles (in the presence of an appropriate catalyst). To maintain stability of the glycogen or phytoglycogen nanoparticles, the pH is preferably between 8 and 10 and optimally between 8 and 9. Hydroxyl reactivity is low in such conditions, and a significant excess of reacting compound (reactant) may be necessary to obtain a significant functionalization. Although derivatization in aqueous environment is often preferable, some reactions (e.g. with alkyl halides) are best conducted in organic solvents such as dimethyl sulfoxide (DMSO), dimethyl formamide (DMF) or pyridine. As will be apparent to one of skill in the art, water-soluble compounds with low toxicity and reactive at relatively mild conditions are particularly suitable.

By way of example, the simplest approach to activate a hydroxyl group of glycogen or phytoglycogen is the introduction of carbonyl groups by selective oxidation of glucose hydroxyl groups at positions of C-2, C-3, C-4 and/or C-6. There is a wide spectrum of redox initiators which can be employed, such as persulfate, periodate (e.g. potassium periodate), bromine, acetic anhydride, (2,2,6,6-tetramethylpiperidin-1-yl)oxyl (TEMPO), Dess-Martin periodinane, etc.

Glycogen and phytoglycogen nanoparticles functionalized with carbonyl groups are readily reactive towards compounds bearing primary or secondary amine groups. This results in imine formation (eq. 1) which can be further reduced to amines with a reducing agent e.g., sodium borohydride (eq. 2). This reduction step provides an amino-product which is more stable than the imine intermediate, and also converts unreacted carbonyls in hydroxyl groups. The elimination of carbonyls significantly reduces the possibility of non-specific interactions of derivatized nanoparticles with non-targeting molecules (e.g. plasma proteins).

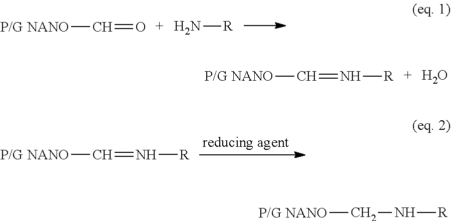

The reaction between carbonyl- and amino-compounds, as well as the reduction step, can be conducted simultaneously in one vessel, with a suitable reducing agent introduced to the same reaction mixture. This reaction is known as direct reductive amination. Here, any reducing agent, which selectively reduces imines in the presence of carbonyl groups (e.g. sodium cyanoborohydride) can be used.

For the preparation of amino-functionalized nanoparticles from carbonyl-functionalized nanoparticles, any ammonium salt or primary or secondary amine-containing compound can be used (e.g., ammonium acetate, ammonium chloride, hydrazine, ethylenediamine, or hexanediamine). This reaction can be conducted in water or aqueous polar organic solvent (e.g. ethyl alcohol, DMSO, or DMF).

Reductive amination of the nanoparticles can be also achieved by the following two step process. First step is allylation, i.e., converting hydroxyls into allyl-groups by reaction with allyl bromide in the presence of a reducing agent (e.g. sodium borohydride). In the second step, the allyl-groups are reacted with a bifunctional aminothiol compound (e.g. aminoethanethiol).

Amino-functionalized nanoparticles are amenable to further modifications. Amino groups are reactive to carbonyl compounds (aldehydes and ketones), carboxylic acids and their derivatives, (e.g. acyl chlorides, esters), succinimidyl esters, isothiocyanates, sulfonyl chlorides, etc.

Degree of substitution depends on the molecular weight and properties (charge, hydrophobicity, etc.) of the molecules to be conjugated. Degree of substitution is expressed as of glucose units derivatized. E.g. if a drug has a molecular weight of 100 Da, and the degree of substitution is 50%, then 1 g of phytoglycogen/glycogen nanoparticles would carry 0.28 g of the drug. For small molecules (<100 Da) a degree of substitution >30% was generally achieved, going as high as 100% for methyl groups. Larger molecules (which cannot penetrate the pore structure of the particles) can be conjugated only at the surface of the phytoglycogen/glycogen nanoparticles, and the degree of substitution is lower, generally 0.1-2.0%.

Modified nanoparticles may further be conjugated to one or more compounds selected from biomolecules, small molecules, therapeutic agents, pharmaceutically active moieties, macromolecules, diagnostic labels, to name a few, as well as various combinations of the above. Nanoparticles can be further modified with specific tissue targeting molecules, such as folic acid, antibodies, aptamers, proteins, lipoproteins, hormones, charged molecules, polysaccharides, and low-molecular-weight ligands. Two or more different chemical compounds can be used to produce multifunctional derivatives. For example, one chemical compound can be selected from the list of specific binding biomolecules, such as antibody and aptamers, while the second modifier would be selected from the list of diagnostic labels or therapeutics.

A chemical compound bearing a functional group capable of binding to an amine-group of modified glycogen or phytoglycogen nanoparticles or the hydroxyl groups of unmodified nanoparticles can be directly attached to functionalized phytoglycogen/glycogen nanoparticles. However, for some applications chemical compounds may be attached via a polymer spacer or a “linker”. These can be homo- or hetero-bifunctional linkers bearing functional groups such as amino, carbonyl, sulfhydryl, succimidyl, maleimidyl, and isocyanate, (e.g. diaminohexane), ethylene glycobis(sulfosuccimidylsuccinate), disulfosuccimidyl tartarate, dithiobis(sulfosuccimidylpropionate), aminoethanethiol, etc.

The location of molecules conjugated to phytoglycogen/glycogen nanoparticles depends on the molecular weight of the molecule. Small molecules (MW<100 Da) can enter the particle structure and, therefore, are located within and at the particle surface. Molecules with MW >100 Da are located predominantly at the particle surface.

When phytoglycogen nanoparticles are internalized conjugated molecules are released by cellular hydrolases. The rate of release can be controlled by the degree of phytoglycogen derivatization by small molecules, e.g, methylation, hydroxupropylation, (which affect the affinity of hydrolases to polysaccharide chain and, therefore, the rate of hydrolysis).

In one embodiment, the glycogen or phytoglycogen nanoparticles are cationized, in one embodiment, amine-modified, but are not further conjugated to another molecule.

In one embodiment, the glycogen or phytoglycogen nanoparticles are cationized, in one embodiment, amine-modified, and are further conjugated to a pharmaceutically useful moiety selected from a hydrophobicity modifier, a pharmacokinetic modifier, a biologically active modifiers or a detectable modifier.

In one embodiment, there is provided an immunosuppressive composition comprising substantially monodisperse glycogen or phytoglycogen nanoparticles; and a pharmaceutically acceptable carrier. In one embodiment, the glycogen or phytoglycogen nanoparticles are bound to at least one molecule that induces, enhances or suppresses an immune response in a subject. In one embodiment, the molecule is an immune suppressant.

Compounds and compositions as described herein are useful as immunosuppressives.

As demonstrated in the Examples, cationized glycogen/phytoglycogen nanoparticles and, in particular, amine-modified nanoparticles act as a suppressor of the type 1 IFN-dependent innate antiviral response.

The type I interferon system consists of type I interferons (IFNs), the signaling pathways triggered by IFNs binding their receptors, the transcription factors activated by these pathways, the genes whose expression is altered as a result of transcription factor activation (called interferon stimulated genes or ISGs), and finally the change in cellular function. The function that led to the discovery of the IFNs was their capacity to establish an ‘antiviral state’ in infected cells as well as neighbouring uninfected cells. IFNs are divided into two classes, type I and type II. Classically, type I IFNs are involved in innate antiviral mechanisms, whereas type II IFNs promote adaptive immunity. Type I interferons have been shown to inhibit every stage of viral replication. This includes viral entry and uncoating, transcription, RNA stability, initiation of translation, maturation, assembly and release.

The antiviral effects of type I IFNs are initiated by the binding of interferon to its cognate receptor found on the surface of all nucleated cells. The IFNa/b signaling pathway in mammals involves five major steps. IFN binding causes dimerization of the IFN receptor (1). This receptor association triggers signaling through the Janus kinase (Jak)/Signal transducers and activators of transcription (STAT) pathway by activating Janus kinases, Jak1 and Tyk2 (2). These tyrosine-kinases phosphorylate STAT1 and STAT2, which are associated with the IFN receptor, leading to their activation and dimer formation (3). The activated STAT 1-2 heterodimers translocate to the nucleus (4) and associate with p48 (IRF-9) to form ISGF3, a transcription factor which binds to interferon-stimulated response element (ISRE) sequences in the promoter regions of interferon stimulated genes (5; ISGs) (Stark et al., 1998). Type I interferons generally induce the same set of genes within the same cell type. It is these ISGs that accumulate in the target cell, establishing an ‘antiviral state’.

The primary purpose of type I interferons is to stimulate the expression of ISGs, which in turn confer an antiviral state within uninfected cells. Interferon inducible factors tend to either limit virus replication directly or regulate cell cycle and cell death. Programmed cell death or apoptosis, which is stimulated by some ISGs, is considered a strategy to control viral replication. Many IFN stimulated proteins are enzymes that are expressed in an inactive form until exposed to dsRNA, ensuring an antiviral state that remains dormant and therefore harmless until the cell is infected.

Innate immunity is highly conserved between vertebrates. With reference to the Examples, the present inventors have demonstrated that glycogen and phytoglycogen nanoparticles in both an unmodified or cationized form and, in particular, when amine modified are capable of suppressing type I interferon responses in vertebrate cells. Accordingly, there is provided immunosuppressive compounds and compositions for suppressing type I interferon responses.

While autoimmune diseases may be associated with genetic predisposition, autoimmunity may only be triggered after stimulation by environmental factors, including viral infections. Immunosuppressives of the IFN-dependent antiviral response can be useful in the treatment or prevention of a number of diseases or conditions as detailed further below. Sepsis is a life-threatening condition that arises when the body's response to infection injures its own tissues and organs. Severe sepsis is one of the most common diagnosies in patients admitted to the intensive care unit (ICU), affecting >750,000 patients/yearly in the United States, and costing >$17 billion per year. Sepsis is usually treated with intravenous fluids and antibiotics, however, there are no approved drugs for use to block cytokine production in order to reduce sepsis symptoms in patients. There remains a need for additional treatment options for sepsis. In one embodiment, there is provided a novel method of treating sepsis comprising blocking type I interferon production using compounds or compositions as described herein. In one embodiment, compounds and compositions as described herein are introduced systemically into severe sepsis patients to reduce systemic cytokine production.

In one embodiment, the immunosuppressive composition is used in preventing rejection of a transplanted organ or tissue.

In one embodiment, the immunosuppressive composition is used in the treatment of an inflammatory disease e.g irritable bowel disorder.

In another aspect, the immunosuppressive compounds and compositions described herein are used in treating an autoimmune disease. In various embodiments the autoimmune disease is rheumatoid arthritis, multiple sclerosis, myasthenia gravis, systemic lupus erythematosus, sarcoidosis, focal segmental glomerulosclerosis, Crohn's disease, Behcet's Disease, pemphigus, and ulcerative colitis.

In another embodiment, compounds and compositions described herein are used in the treatment of pain, namely inflammatory pain.

Aside from being used alone, immunomodulators as described herein may be used in combination with other therapeutics such as antimicrobial or anticancer agents, vaccines or other immunomodulators.

In one embodiment the immunosuppresive compounds and compositions described herein may be used in combination with transfection formulations and, in particular embodiments, transfection using viral vectors. In one embodiment, compounds and compositions of the present invention may be administered concurrently, or shortly before or after gene therapy. Viral-vector based gene therapies may be preceded or followed by immunosuppressive therapy to prevent immune reactions to the virus. For example, alipogene tiparvovec (marketed under the trade name Glybera) is approved by the European Medicines Agency for the treatment of lipoprotein lipase deficiency (LPLD). Glybera uses an adeno-associated virus vector that delivers a copy of the human lipoprotein lipase (LPL) gene to muscle cells. For three days before treatment, which is administered by injection, and for 12 weeks after injection, the patient is administered immunosuppressive treatment.

Thus, in one embodiment, there is provided a combination therapy of a viral-vector based gene therapy and glycogen or phytoglycogen nanoparticles as described herein.

In another aspect, the immunosuppressive compounds and compositions described herein are used in the manufacture of vaccines. Vaccine production involves several stages, first of which is the generation of the antigen itself. In the case of viral vaccines, viruses are generated by infecting cultured cells. The yield from such procedures is typically low due to the cells' innate immune systems and/or responses as described herein, which tend to fight the viruses. By suppressing the immune systems and/or responses of the cells, the viruses can replicate more easily resulting in an increased yield and/or allowing for the production of higher virus titres.

Thus, in one embodiment, there is provided use of the immunosuppressive compounds and compositions described herein for manufacturing vaccines, in particular, enhancing the manufacture of a viral vaccine by increasing or improving the growth and culturing of viruses and viral antigens. For example in one preferred embodiment, the glycogen or phytoglycogen nanoparticles described herein is useful in the manufacture of infectious pancreatic necrosis virus (IPNV) vaccine using a CHSE-214 (Chinook salmon embryo) cell line.

In one embodiment, a limiting factor in vaccine development using cell lines as a source for a virus is the IFN pathway reducing virus production. With the addition of PHX-NH2 at low levels (for example, 0.005 ng/mL), virus titres increase compared to cells infected without PHX-NH2 present.

Examples of viruses and cell lines used in vaccine manufacturing which may be enhanced using the glycogen or phytoglycogen nanoparticles described herein are listed in Table 1.

TABLE 1
Example Viruses and Cell Lines for PHX mediated Viral growth
enhancement

| | Cell Lines Used for Vaccine |
|---|---|
| Viruses: | Manufacturing: |
| Fish: | |
| Infectious Pancreatic Necrosis | |
| Virus (IPNV) | |
| Red Sea Bream Iridovirus (RSIV) | |
| Rock bream Iridovirus (RBIV) | |
| Infectious Salmon anemia virus | |
| (ISAV) | |
| Nervous Necrosis Virus (NNV) | |
| Dogs: | |
| Canine Parvovirus (CPV) | BF-2 (Bluegill) |
| Canine Distemper Virus (CDV) | GF-1 (Hamilton Grouper) |
| Canine Adenovirus-2 (CAV-2) | RTgillW-1 (Rainbow Trout) |
| Canine Rabies Virus | MFF-8C1 (Mandarin Fish Fry) |
| Canine Parainfluenza Virus | CHSE-214 (Chinook salmon |
| Canine Influenza Virus (CIV) | embryo) |
| Canine Enteric Coronavirus | |
| Cats: | |
| Feline Herpesvirus 1 (FHV-1) | A-72 (Canine carcinoma) |
| Feline Calicivirus (FCV) | FK (Feline Kidney) |
| Feline Panleukopenia Virus | MDBK (Madin-Darbey Bovine |
| (FPV) | Kidney Epithelial) |
| Feline Rabies Virus | MDCK (Madin-Darby Canine |
| Feline Leukemia Virus (FeLV) | Kidney Epithelial) |
| Pigs: | |
| Porcine circovirus type 2 (PCV2) | WI-38 (Human diploid lung |
| Porcine Reproductive and | fibroblasts) |
| respiratory syndrome virus | MRC-5 (Human diploid lung) |
| (PRRSV) | HEK-293 (Human embryonic |
| Swine influenza virus | kidney) |
| Classical swine fever virus | Per-C6 (Human embryonic |
| Pseudorabies virus | retina) |
| Cattle: | |
| Bovine parainfluenza virus | |
| Bovine respiratory syncytial virus | |
| (BRSV) | |
| Infectious Bovie Rhinotracheitis | |
| virus (IBRV) | |
| Bovine Viral Diarrhea Virus | |
| (BVDV) | |
| Ovine Infectious | |
| Encephalomyelitis Virus | |
| Poultry: | |
| Chicken anemia virus | |
| Infectious bursal disease virus | |
| Newcastle Disease Virus (NDV) | |
| Avian Influenza Virus (H5N1) | |
| Avian Encephalomyelitis Virus | |
| Derzsy's disease goose | |
| parvovirus | |
| Duck hepatitis | |
| Duck Herpes Virus 1 | |
| Fowl pox virus | |
| Hemorrhagic enteritis virus | |
| Infectious bronchitis virus (IBV) | |
| Inclusion body hepatitis virus | |
| Infectious Laryngotracheitis virus | |
| (ILTV) | |
| Marek's disease virus (MDV) | |
| Egg drop syndrome virus | |
| Avian infectious bursal disease | |
| virus | |
| Avian reovirus | |
| Avian pneumovirus | |
| Turkey hemorrhagic enteritis | |
| virus | |
| Turkey rhinotracheitis virus | |
| Human: | |
| Varicella zoster virus (VZV) | |
| Hepatitis A Virus (HAV) | |
| Hepatitis B Virus (HBV) | |
| Human Papilloma virus (HPV) | |
| Influenza A/B virus (IAV/IBV) | |
| Japanese encephalitis virus | |
| (JEV) | |
| Measles virus | |
| Mumps virus | |
| Poliomyelitis virus | |
| Rabies virus | |
| Rotavirus | |
| Rubella virus | |
| Yellow fever virus (YFV) | |
| Variola virus | |

### Formulation and Administration

The nanoparticles of the invention may also be admixed, encapsulated, or otherwise associated with other molecules, molecule structures or mixtures of compounds and may be combined with any pharmaceutically acceptable carrier or excipient. As used herein, a “pharmaceutically carrier” or “excipient” can be a pharmaceutically acceptable solvent, suspending agent or any other pharmacologically inert vehicle for delivering functionalized glycogen or phytoglycogen nanoparticles, whether alone or conjugated to a biologically active or diagnostically useful molecule, to an animal. The excipient may be liquid or solid and is selected, with the planned manner of administration in mind, so as to provide for the desired bulk, consistency, etc., when combined with glycogen or phytoglycogen nanoparticles and the other components of a given pharmaceutical composition. Examples of pharmaceutically acceptable carriers include one or more of water, saline, phosphate buffered saline, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the pharmacological agent.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers, finely divided solid carriers, or both, and then, if necessary, shaping the product (e.g., into a specific particle size for delivery).

For the purposes of formulating pharmaceutical compositions, monodisperse glycogen and phytoglycogen nanoparticles prepared as taught herein, may be provided in a dried particulate/powder form or may be dissolved e.g in an aqueous solution. Where a low viscosity is desired, the glycogen and phytoglycogen nanoparticles may suitably be used in formulations in a concentration of up to about 25% w/w. In applications where a high viscosity is desirable, the glycogen and phytoglycogen nanoparticles may be used in formulations in concentrations above about 25% w/w. In applications where a gel or semi-solid is desirable, concentrations up to about 35% w/w can be used.

The pharmaceutical compositions of the present invention may be administered in a number of ways depending upon whether local or systemic treatment is desired and upon the area to be treated. Without limiting the generality of the foregoing, the route of administration may be topical, e.g. administration to the skin or by inhalation or in the form of opthalmic or otic compositions; enteral, such as orally (including, although not limited to in the form of tablets, capsules or drops) or in the form of a suppository; or parenteral, including e.g. subcutaenous, intravenous, intra-arterial or intra-muscular. Suitably, compounds and composition for treatment of sepsis are administered systemically.

In one embodiment, the pharmaceutical composition is a topical formulation for application to the skin, for transdermal delivery. The monodisperse nanoparticles disclosed herein are particularly useful as film-forming agents. Because the nanoparticles are monodisperse, uniform close-packed films are possible. The compositions form stable films with low water activity. Accordingly, when chemically modified, they may be used to attach and carry bio-actives across the skin. In various embodiments, the topical formulation may be in the form of a gel, cream, lotion or ointment.

In another embodiment, the pharmaceutical compositions of the present invention are in the form of an implant. Suitably, these implants may be biocompatible, meaning that they will have no significant adverse effects on cells, tissue or in vivo function. Suitably, these implants may be bioresorbable or biodegradable (in whole or in part). Examples include, without being limited to, tissue engineering scaffolds.

In another embodiment, the glycogen or phytoglycogen nanoparticles can be part of topical (medical) formulations e.g., lotions, ointments, and creams.

### EXAMPLES

### Example 1. Manufacture of Phytoglycogen from Sweet Corn Kernels

1 kg of frozen sweet corn kernels (75% moisture content) was mixed with 2 L of deionized water at 20° C. and was pulverized in a blender at 3000 rpm for 3 min. Mush was centrifuged at 12,000×g for 15 min at 4° C. The combined supernatant fraction was subjected to cross flow filtration (CFF) using a membrane filter with 0.1 μm pore size. The filtrate was further purified by a batch diafiltration using membrane with MWCO of 500 kDa and at RT and diavolume of 6. (Diavolume is the ratio of total mQ water volume introduced to the operation during diafiltration to retentate volume.)

The retentate fraction was mixed with 2.5 volumes of 95% ethanol and centrifuged at 8,000×g for 10 min at 4° C. The retentate was mixed with 2.5 volumes of 95% ethanol and centrifuged at 8,000×g for 10 min at 4° C. The pellet containing phytoglycogen was dried in an oven at 50° C. for 24 h and then milled to 45 mesh. The weight of the dried phytoglycogen was 97 g.

According to dynamic light scattering (DLS) measurements, the phytoglycogen nanoparticles produced had particle size diameter of 83.0 nm and a polydispersity index of 0.081.

### Example 2. Modification of Phytoglycogen/Glycogen Nanoparticles

Examples of chemically modified phytoglycogen/glycogen nanoparticles synthesized for different applications and their degree of substitution are listed in Table 2.

TABLE 2

| Product | DS* | Chemistry/intermediate |
|---|---|---|
| Amino-P/G Nano | 0.1 | Carbonyl-P/G Nano |
| Quab151⁶-P/G Nano | 1.00 | Epoxide |
| Castor/Quab151-P/G Nano | 0.025 | Double modification |
| | (Quab151) | |
| | 0.001 | |
| | (Castor) | |
| Cy5.5-P/G Nano | 0.002 | succinic anhydride |

P/G Nano - phytoglycogen/glycogen nanoparticle
Castor - QUAB151 - 2,3-epoxypropyltrimethylammonium chloride
*DS - degree of substitution measured

### Amination of Phytoglycogen Nanoparticles

200 mg of phytoglycogen, obtained as described in Example 1, was dissolved in 2 mL DMSO and 250 mg of dry powdered NaOH was added to solution. The P phytoglycogen was permitted to stir for 15 minutes in basic DMSO before 1.5 mL 2-romoethylamine hydrobromide was added. The reaction was allowed to proceed for 10 minutes, after which an additional 0.5 mL DMSO was added to the reaction vial. The reaction was then allowed to continue for 4 hours at room temperature. After 4 hours, sample was diluted to 10 mL with deionized water and, to this solution, two volumes of ethanol were added to precipitate aminated nanoparticles. Ethanol precipitation was repeated for a total of three times. The resulting sample pellet was dispersed and dried in ether to give a powdered final product. By peak integration of a ¹H-NMR spectrum, 5.2 mol % of the glucose units were aminated.

100 g of phytoglycogen was dispersed in 500 ml of 0.45 M NaOH solution in water then 200 g of 2,3-epoxypropyltrimethylammonium chloride (90%, Sigma-Aldrich) was added to the mixture. The mixture was stirred for 12 h at 40° C. After the reaction completed, an excess amount of 95% ethyl alcohol was added to stop the reaction and precipitate the product. The precipitate was dispersed in 500 ml of water, neutralized with 0.2M HCl and precipitated again with 95% alcohol. The product was washed three times using this dispersion in water-precipitation operation, then dried in an oven at 60° C. for 18 h. The dried modified phytoglycogen was milled to 200 mesh powder then placed on a filter on a vacuum filtration funnel and washed with 70% alcohol twice and 95% alcohol once under vacuum to remove the cationization reagent. The cake was dried under 50° C. for 18 hours. The DS of the product was assessed using NMR spectroscopy and was found to be 1.072.

### Octenyl Succinic Anhydrite-Modified Phytoglycogen

100.0 g of phytoglycogen produced according to Example 1 was dispersed in 750 mL of de-ionized water in a 2 L glass reaction vessel. The dispersion was constantly stirred and kept at 35° C. 3 mL of octenyl succinic anhydride (OSA, Sigma-Aldrich) was heated to 40° C. and was slowly added into the reaction vessel. The pH was kept constant at 8.5 by adding a 4% NaOH solution to the reaction mix using an automated control system. The reaction was allowed to proceed for 3 h under constant mixing. Then the pH of the mixture was adjusted to 7.0 with 1 M HCl and was mixed with 3 volumes of 95% ethanol and centrifuged at 8,500×g for 15 min at 4° C. The pellet was re-suspended in water, the pH was adjusted to 7.0, and the solution was precipitated and centrifuged using the same conditions twice. Finally, the pellet containing OSA-modified phytoglycogen was dried in an oven at 50° C. for 24 h and then milled to 45 mesh. The degree of substitution determined by NMR spectroscopy was 0.024.

### Example 3 Cytotoxicity of Glycogen/Phytoglycogen in Cell Cultures

The effects of the glycogen/phytoglycogen nanoparticles on cell viability was analyzed to assess cytotoxicity of the particles. Glycogen/Phytoglycogen nanoparticles were extracted from rabbit liver, mussels, and sweet corn using cold-water and isolated as described in Example 1.

Modified particles were prepared by functionalizing phytoglycogen with amino-groups (NH₂), quaternary ammonium groups (Quab151) and OSA3 as described in Example 2.

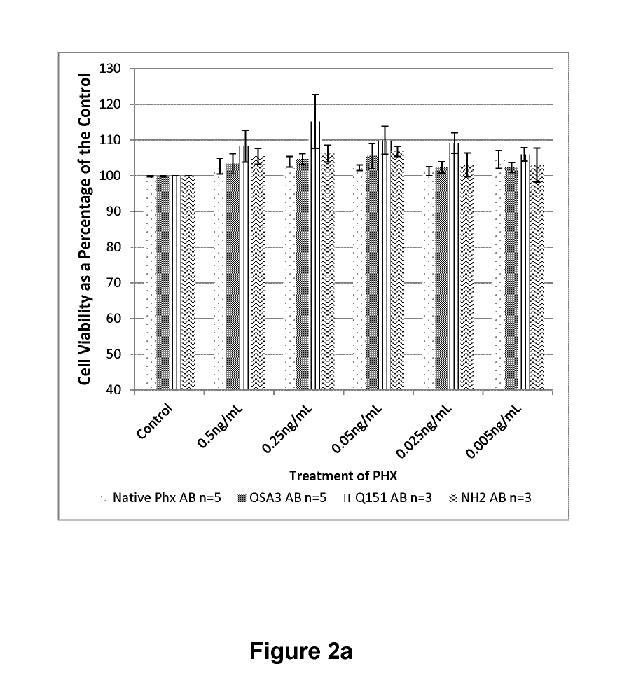

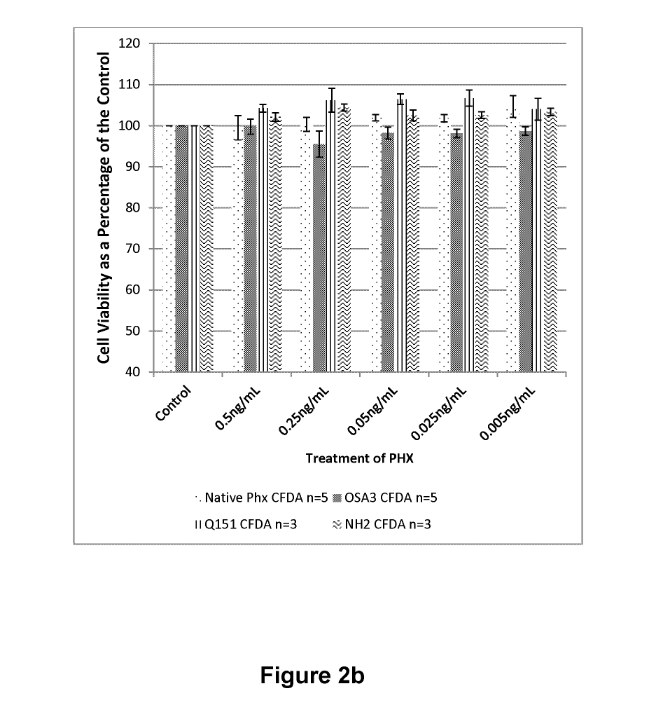

Rainbow trout gill epithelium (RTG-2) cells were treated with unmodified and the modified phytoglycogen nanoparticles for 3 days. After this treatment, the media was removed and cellular metabolism and membrane integrity were measured. To measure changes in cell viability two fluorescence indicator dyes were used, alamar blue (ThermoFisher) (FIG. 2a) and CFDA-AM (Thermofisher) (FIG. 2b); these dyes measure cell metabolism and membrane integrity respectively. For these dyes, more fluorescence indicates more viable cells.

No changes in either parameter were significant compared to untreated control cells for any treatment. The results are presented in FIG. 2. None of the assays detected any cytotoxicity effects in cells after 72 h incubation in the presence of phytoglycogen or its derivatives at concentrations of 0.1-10 mg/ml.

### Example 4. Changes in Innate Immune Genes Expression Levels

Experiments were conducted to determine whether amine (NH₂) functionalized monodisperse compositions of phytoglycogen nanoparticles (PHX-NH₂) have immunosuppressive activity. More specifically, the ability of such compositions to inhibit the type I IFN-dependent innate antiviral response was investigated. This IFN-mediated response was studied by measuring changes in gene expression at the transcript level by quantitative (q)RT-PCR and changes in establishment of an antiviral state by using a Cytopathic Effect (CPE) Assay to quantify virus-induced syncytia formation. If such compositions are able to block IFN-mediated responses, transcript levels for IFN-stimulated genes (ISGs) would decrease, and the IFN-induced antiviral state would be compromised, thus resulting in more CPE (syncytia formation).

Three salmonid cell lines obtained from N. Bols (University of Waterloo) were used in this study, RTG-2 (rainbow trout gonadal origin), RTgill W-1 (rainbow trout gill origin) and CHSE-214 (chinook salmon embryonal origin). RTG-2, RTgill W-1 and CHSE-214 were all routinely cultured at 20° C. in 75 cm² plastic tissue culture flasks (BD Falcon, Bedford, Mass.) with Leibovitz's L-15 media supplemented with 10% fetal bovine serum (FBS) and 1% penicillin/streptomycin (P/S).

For this study, two fish viruses were used. Chum salmon reovirus (CSV) was propagated on monolayers of Chinook Salmon Embryonic (CHSE-214) cells. Viral hemorraghic septicima (VHSV)-IVb was propagated using epithelioma papulosum cyprinid (EPC) cells. Both virus preparations, virus containing media (L-15 with 5% FBS) was filtered through 0.45 um filter after 4-7 days (when complete CPE was observed). For short term storage virus preparations was frozen at −20° C. and long term storage at −80° C. Tissue culture infectious dose (TCID)₅₀/mL values (50% tissue culture infective dose) were determined using the Reed and Muench method by titering VHSV-IVb preparations on EPC cells and CSV preparations on CHSE-214 cells.

PHX-NH₂ (was pre-mixed with virus CSV 1.995×10⁴, PHX—NH₂ 0.5 ng/mL and VHSV 2.5×10⁵ TCID50, PHX—NH₂ 0.5 ng/mL and 0.05 ng/mL; or PBS alone (without magnesium and calcium) and rocked at room temperature for 2 h. For VHSV, after 2 h an equal volume of 2× L-15 with 10% FBS was added to each sample. For CSV, because the stock titre was not high enough to dilute first in PBS all incubations were performed in full media and an equal volume 1×L-15 was added after rocking. RTG-2 cells were infected with CSV for 24 hours and RTgill W-1 cells were infected with VHSV at 17 degrees for 72 hours.

RNA extraction using the GenElute Mammalian total RNA miniprep kit (Sigma Aldrich) following the manufacturer's instructions including treating the RNA with an on-column DNase I digestion set (Sigma Aldrich). cDNA synthesis was performed using an iScript cDNA synthesis kit (Bio-rad) using 1 ug of RNA, 4 uL of iScript and up to 20 uL DNA quality water in each reaction. The cDNA was diluted 1 in 10 in nuclease-free water prior to qPCR reactions.

All PCR reactions contained: 2 pL of diluted cDNA, 2× SsoFast EvaGreen Supermix (Bio-Rad), 0.2 μm forward primer, 0.2 μM reverse primer and nuclease-free water to a total volume of 10 μL (the housekeeping gene actin primers were at 0.1 μM). The qPCR program was 98° C. 2 mins, 40 Cycles of 98° C. 5 s, 55° C. 10 s and 95° C. for 10 s. A melting curve was completed from 65° C. to 95° C. with a read every 5 s. Gene expression was normalized to the housekeeping gene (β actin) and expressed as a fold change over the untreated control group.

Unmodified and amine-functionalized monodisperse phytoglycogen nanoparticles (0.5 ng/mL) were pre-mixed with CSV (TCID_{50/mL} 6.25×10⁴) in 5% FBS 1×L-15 media and rocked at room temperature for 2 h. After 2 hours the appropriate amount of 5% FBS 1×L-15 was mixed thoroughly and added to each well. CHSE-214 was seeded at 3×10⁴ cells/well in a 96 well plate and left to grow for 24 hours at 20° C. After which, the cells were treated with the pre-mixed nanoparticles and CSV at 17° C. for 3 days.

CHSE-214 were fixed with 75 μL/well of 10% Formalin for 10 mins, rinsed with 100 μL/well PBS and stained with 75 μL/well of 1% Crystal Violet Stain for 10 mins. The wells were then washed twice with 100 μL/well of PBS and at least 5 times with MilliQ Water. Pictures were taken using a Nikon Eclipse TiE microscope with Qi1 camera at 4× magnification. The area covered by syncytia and the total area of the picture were calculated using Nikon NIS element software. % syncytia is the area covered by syncytia/total area*100%. 1 picture was taken/well, each picture was taken of the centre of the well, and there were 6 wells/treatment.

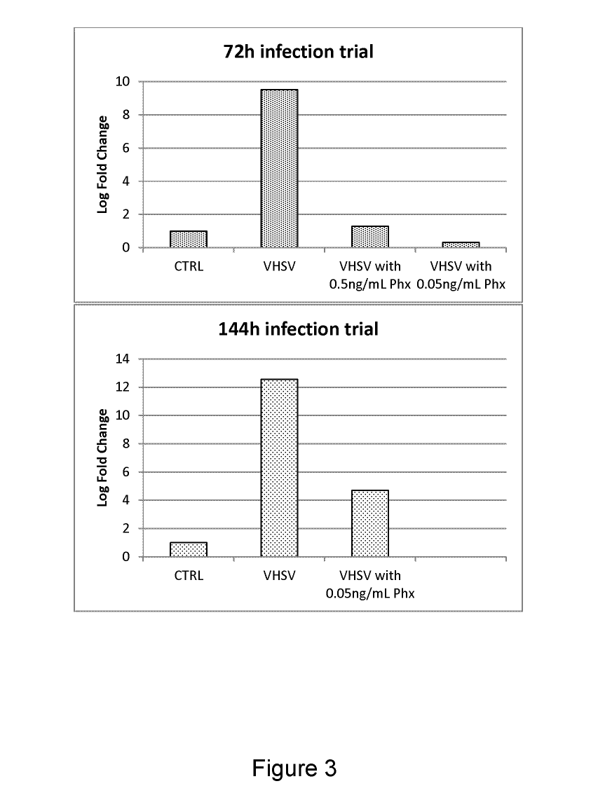

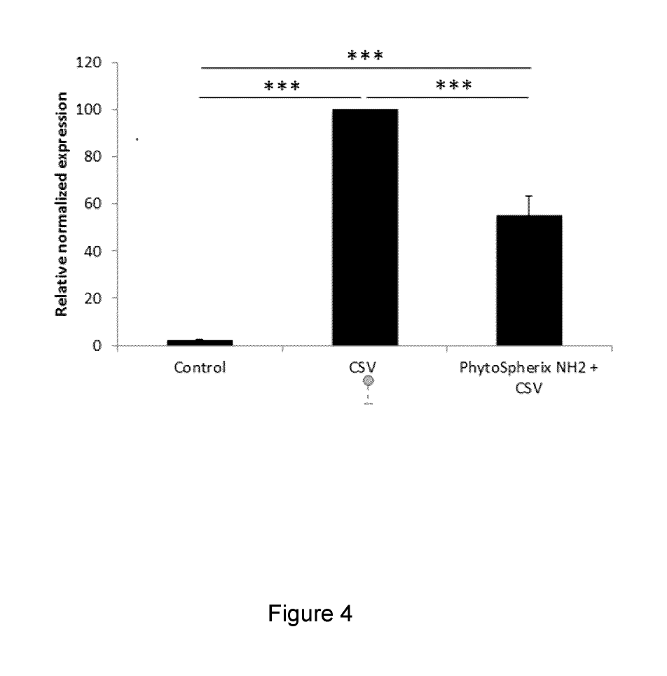

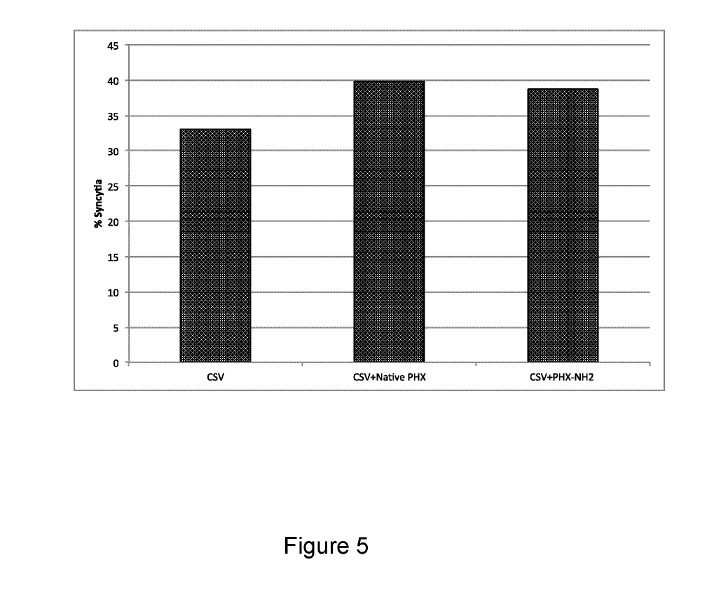

Referring to FIGS. 3 to 5, these data show that PHX-NH2 blocks ISG expression induced by two aquatic viruses, VHSV-IVb and CSV, in two fish cell lines, RTgill-W1 and RTG-2 respectively. It also shows that both native PHX and PHX-NH2 reduced the total antiviral state in CHSE-214 cells, making them more susceptible to CSV-induced CPE (syncytia formation). This data suggests a general suppressive effect of PHX on the type I IFN response.

### Example 5. Internalization of Cy5.5-Labeled Glycogen/Phytoglycogen Particles by TCP-1 Monocytes

Conjugation of a near-infrared fluorescent dye (Cy5.5) to the particles used in this study enabled analysis of nanoparticle uptake by confocal fluorescence microscopy. Cy5.5-labeled glycogen/phytoglycogen particles were produced as described below.

100 mg of polysaccharide nanoparticles, produced according to Example 1, was suspended in 20 mL of 0.1 M Sodium bicarbonate buffer, pH 8.4. With a temperature probe in a control vial (containing 0.1 M Sodium bicarbonate buffer), the reaction vessel containing the solution was wrapped in aluminium foil and placed on a hot plate at 35° C. 1 mg Cy5.5-NHS ester (Lumiprobe Corp.) was suspended in 4 mL DMF. During a 1 h period, Cy5.5-NHS ester was added in 1-mL aliquots. The pH of solution was constantly checked before and after addition, adjusting to 8.4 with the addition of a 2 M HCl solution. After the final aliquot of Cy5.5-NHS ester in DMF was added, the pH was monitored and adjusted as needed. The reaction was allowed to proceed for 2 h further, after which the pH was adjusted to 4.0 with a 2 M HCl solution as aforementioned.

To the acidified solution containing the resulting polysaccharide nanoparticle-Cy5.5 conjugate was added 2 volumes of ethanol. This solution was cooled to 4° C. and centrifuged at 6000 rpm for 15 minutes. After centrifugation, the supernatant was poured off and the pellet was resuspended in 15 mL deionized water. 2 volumes of ethanol was added to the resuspended pellet and it was cooled and centrifuged as before. This was repeated one time further until the supernatant that was poured off was clear and colourless. The pellet was resuspended a final time in 10 mL anhydrous Diethyl ether via use of a homogenizer. The resulting conjugate was rendered by evaporating to dryness with trace heat.

MCP-1 cells were incubated with Cy5.5-labeled glycogen/phytoglycogen particles at a concentration of 1 mg/ml at 4° C. (negative control) and 37° C. for 0.5, 2, 6 and 24 h. Then cells were washed with PBS, fixed in 10% Buffered Formalin Solution and washed again with PBS. Then fixed cells were stained with DAPI (nucleus) and AF488 (cell membrane). Internalization of glycogen/phytoglycogen particles was assessed by Olympus Fluoview FV1000 Laser Scanning Confocal Microscope.

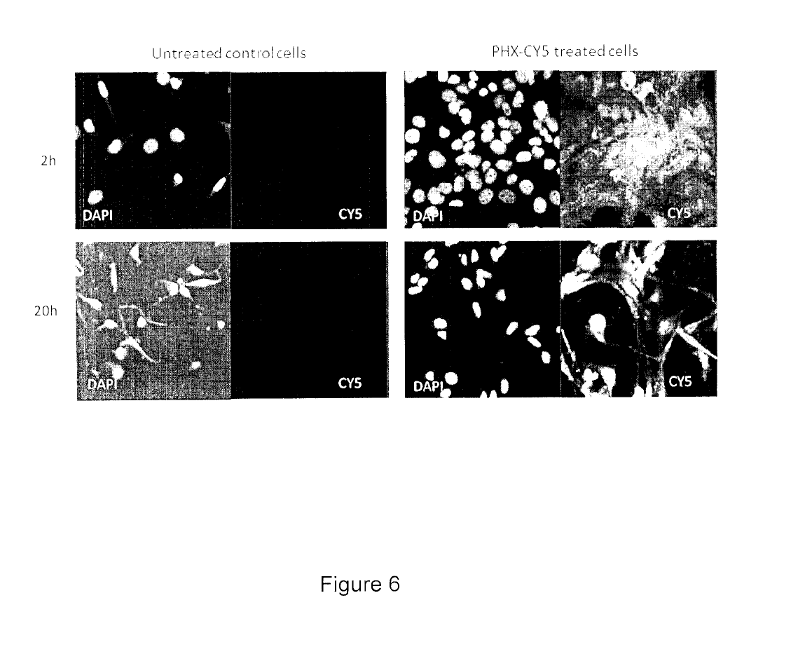

Incubation at 4° C. when endocytotic and phagocytotic processes are no longer active, did not result in any particles associated with THP-1 cells (FIG. 6). This confirmed, that there was no accumulation of the nanoparticles by THP-1 cells due to the surface binding. In contrast, incubation 37° C. for over 6 hours revealed considerable accumulation of Cy5.5-labeled glycogen/phytoglycogen particles in cell cytoplasm (FIG. 6). However, there was very low uptake in the time interval of 0.5-2 h.

### Example 6. Pharmacokinetic (PK) Profile in Naive Mouse after Injection of Cy5.5-Phytoglycogen Conjugate

Cy5.5 labeled phytoglycogen (0.08 μM Cy5.5/mg) was synthesized as described in Example 2.

Nude CD-1 mice (n=3), 18-20 grams were injected with Cy5.5-Phytoglycogen dispersed in PBS at a dose of 300 mg/kg mice. Small blood samples (50 μl) were collected from the mouse (submandibular vein) using heparinized tubes at multiple time intervals (15 mins, 1 h, 2 h, 6 h and 24 h). These time points were analyzed by fluorescence using a cytofluorimeter plate reader. Nanoparticle concentration was interpolated using a standard curve consisting of known concentrations of Cy5.5-Phytospherix diluted in blood.

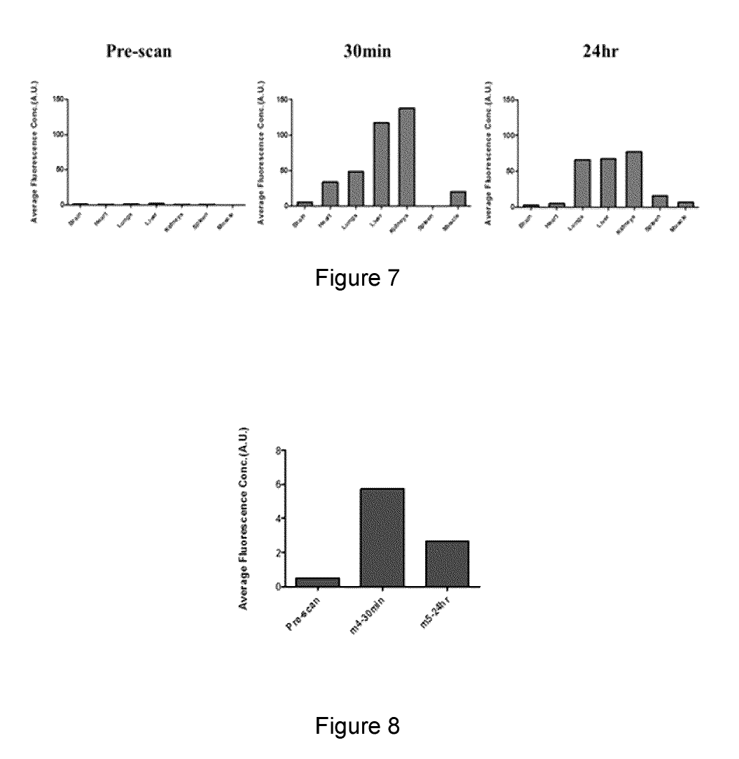

As can be seen from FIG. 7 Cy5.5-phytoglycogen concentration in blood decreased over the time in exponential manner and was eliminated by 24 hrs. The elimination half-life was determined (calculated) to be 2 h. Half-life refers to the period of time required by the body to reduce the initial blood concentration of the compound by 50%.

All optical imaging experiments were performed using a small-animal time-domain eXplore Optix MX2 pre-clinical imager, and images were analyzed or reconstructed as fluorescence concentration maps using ART Optix Optiview analysis software 2.0 (Advanced Research Technologies, Montreal, QC). A 670-nm pulsed laser diode at a repetition frequency of 80 MHz and a time resolution of 12 ps light pulse was used for excitation. The fluorescence emission at 700 nm was collected by a highly sensitive time-correlated single photon counting system and detected through a fast photomultiplier tube.

Cy5.5 labeled phytoglycogen (0.8 μM Cy5.5/mg) was synthesized as described in Example 2.

In naïve animals, in vivo imaging revealed strong signals of Cy5.5-Phytoglycogen in liver, lungs (at all time points), kidney (15 min-6 h), bladder (15 min-6 h), and brain (15 min-2 h) (FIG. 8).

Ex-vivo data at 30 mins and 24 hr confirmed that indeed there was significant uptake of the Cy5.5-Phytoglycogen in lungs and to a lesser degree in brain (FIG. 8). The signal in the brain was highest at earlier time points (30 mins) compared to later time points (24 hrs). Since the Cy5.5-Phytoglycogen nanoparticle is a glucose polymer, it is possible that organs such as brain and lungs, known to be very active in glucose transport, accumulate Cy5.5-Phytoglycogen via glucose transporters.

The in vivo imaging data demonstrated that the liver is mainly responsible for metabolism of the Cy5.5-Phytoglycogen. Furthermore, it is possible that metabolized in liver nanoparticles produce smaller Cy5.5-labeled glucose derivatives that can re-enter the blood stream and then be eliminated through the renal system.

### Example 7. Phytoglycogen Nanoparticles (PHX-NH2) Effect on Chum Salmon Reovirus (CSV) Production, Viral Hemorraghic Septicemia Virus (VHSV)-IVb Production, and Infectious Pancreatic Necrosis Virus (IPNV) Production

### Chum Salmon Reovirus (CSV)

Plating CHSE-214: CHSE-214 (Oncorhynchus tshawytscha) was seeded at 3.2×10⁵ cells/well in 12-well plates and left to grow for 24 hours in Leibovitz's L-15 media with 10% fetal bovine serum (FBS), 1% penicillin/streptomycin (P/S) prior to treatment to allow for cell reattachment. Cells were then treated with PhytoSpherix (PHX-NH₂) alone or with CSV using a 2 hr rocking mixture.

Rocking CSV and PHX-NH2 for 2 hrs:

The following mixtures were rocked for 2 hrs (A) Control: 250 uL of treatment media (TM) comprising L-15 with 5% FBS, and 1% P/S; (B) CSV only: 150 uL of TM and 100 uL of CSV; and (C) CSV with PHX-NH₂: 125 uL TM with 25 uL of 0.1 ng/mL PHX-NH₂ (for a final concentration of 0.005 ng/mL) and 100 uL of CSV. The final CSV tissue culture infectious dose (TCID_{50/mL}) was 3.14×10⁴. PHX—NH₂ was used at a concentration of 0.005 ng/mL. After 2 hrs, 250 uL of TM was added to every microcentrifuge tube and mixed thoroughly by pipette. 500 uL from each microcentrifuge tube was added to individual wells and left to incubate at 17° C. degree incubator for approximately 7 days. The media was then collected and stored at −20 degrees until TCID_{50/mL} was calculated as described below.

CSV TCID_{50/mL} determination using CHSE-214: CHSE-214 were seeded at 3×10⁴ cells/well into 96 well plates and the cells were left to grow for 24 hours in L-15 with 10% FBS, 1% P/S. Cells were then infected with dilutions of virus alone or virus+PHX-NH₂ in dilutions ranging from (10¹-10¹¹ in TM). TM alone was used on the control wells. These plates were incubated at 17° C. for 7 days. After 7 days, the TCID50/mL was determined using the Reed Muench method (1).

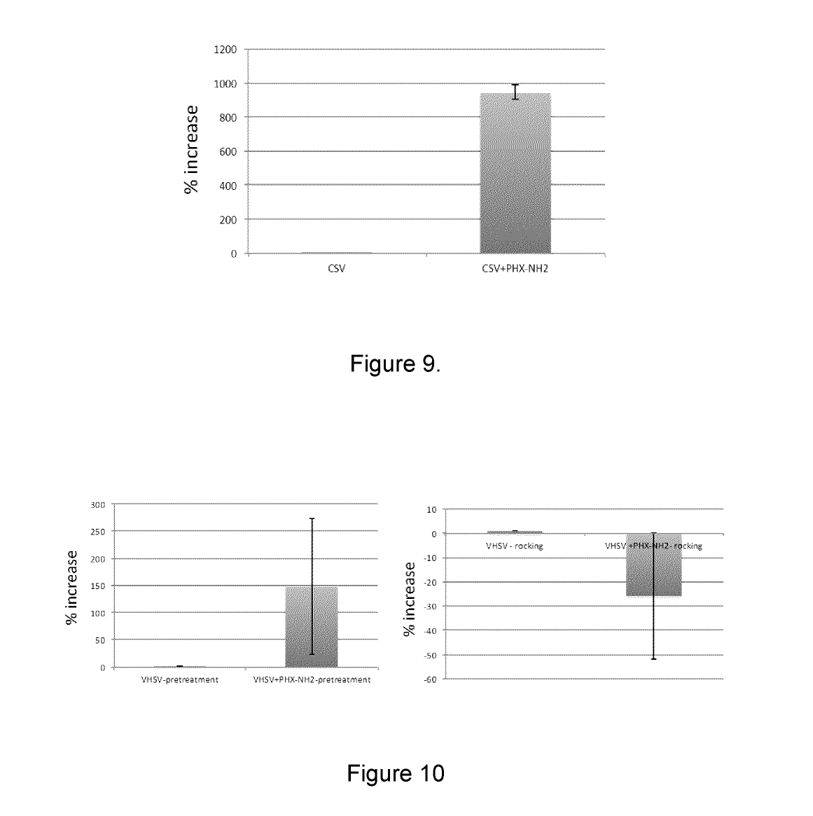

The results are shown in FIG. 9.

Viral Hemorraghic Septicemia Virus (VHSV)-IVb

Plating EPC:

EPC (Epithelioma papulosum cyprinid) were seeded at 5×10⁵ cells/well in 12-well plates and left to grow for 24 hours in L-15 with 10% FBS, 1% P/S prior to treatment to allow for cell reattachment. Cells were treated with PHX-NH₂ using two different methods a) 1 hr pretreatment or b) 2 hr rocking mixture with VHSV as described below. All treatments used a VHSV-IVb stock with a TCID_{50/mL} of 1.72×10⁸ and PHX—NH₂ was used at a concentration of 0.005 ng/mL.

a. Pretreatment with PHX-NH2 for 1 hr:

Cells were treated with (i) L-15 supplemented with 5% FBS, 1% P/S (treatment media) or (ii) 475 uL of media and 25 uL of 0.1 ng/mL PHX-NH₂ (final concentration of 0.005 ng/mL). Cells were incubated for 1 h at 20° C. After 1 hr the media was removed and replaced with the following. (A) Control well: 500 uL of TM; (B) VHSV with PHX-NH₂: 475 uL of TM with 25 uL of 0.1 ng/mL PHX-NH₂ and 50 uL of VHSV; and (C) VHSV alone: 450 uL of TM and 50 uL of VHSV. The plate was then placed in the 17° C. for 7 days. The media was stored at −20° C. until TCID_{50/mL} was calculated as described below.

b. Rocking VHSV and PHX-NH2 for 2 hrs:

The following mixtures were rocked for 2 hrs: (A) Control=250 uL of TM, (B) VHSV alone: 200 uL of TM and 50 uL of VHSV; and (C) VHSV with PHX-NH2: 175 uL of TM with 25 uL of 0.1 ng/mL PHX-NH2 and 50 uL of VHSV. After 2 hrs, 250 uL of TM was added to each mixture and mixed thoroughly by pipette. 500 uL of each mixture was added to individual wells and incubated at 17° C. for 7 days. The media was stored at −20° C. until TCID_{50/mL} was calculated as described below.

VHSV TCID_{50/mL} Determination Using EPC:

For both methods, the TCID_{50/mL} experiments were completed using the same protocol. EPC was seeded at 3×10⁴ cells/well into 96 well plates and left to grow for 24 hours in L-15 with 10% FBS, 1% P/S to allow for cells to reattach. Cells were then infected with dilutions of VHSV alone or VHSV+PHX-NH₂ in dilutions ranging from (10⁻¹-10⁻¹¹ in TM). Regular TM was used on the control wells. Cells were incubated at 17° C. for 7 d, after which the TCID_{50/mL} was determined using the Reed Muench method (1).

The results are shown in FIG. 10.

### Infectious Pancreatic Necrosis Virus (IPNV)

Plating CHSE-214: CHSE-214 (Oncorhynchus tshawytscha) grew until they were approximately 2.4×10⁵ cells/well in 12-well plates in L-15 with 10% FBS, 1% P/S. Cells were then treated with PhytoSpherix (PHX-NH₂) for a 1 hr pretreatment before the addition of IPNV. All treatments used a IPNV stock with a TCID_{50/mL} of 1.09×10⁷ and PHX—NH₂ was used at a concentration of 0.005 ng/mL. Treatment media was L-15 with 2% FBS and 1% P/S.

Pretreatment with PHX-NH2 for 1 hr:

After CHSE-214 cells have reached the desired confluency the media was removed and cells were treated with either PHX-NH2 (50 uL of 0.1 ng/mL PHX-NH2 and 950 uL TM) or media alone (50 uL of phosphate-buffered saline (PBS) and 950 uL TM) for 1 h. After 1 hr, media was removed and cells washed three times with PBS. Cells were then treated with: (A) Control: 950 uL of TM; (B) IPNV alone: 50 uL of PBS and 100 uL of IPNV and 850 uL of TM; and (C) IPNV with PHX-NH2: 850 uL of TM with 50 uL of 0.1 ng/mL PHX-NH2 and 100 uL IPNV. After 2 hours, 100 uL from each well was collected as a day 0 sample and stored at −80° C. Cells were incubated at 14° C. for 7 days, after which the media was collected and stored at −80° C. until the TCID_{50/mL} was calculated as described below.

IPNV TCID_{50/mL} Determination Using CHSE-214:

CHSE-214 was seeded and left to grow until a confluency of 2.4×10⁴ cells/well in 96 well plates in L-15 with 10% FBS, 1% P/S. Cells were then infected with dilutions day 0 and day 7 samples of IPNV alone or IPNV+PHX-NH₂ in dilutions ranging from 10⁻¹-10⁻¹¹ in TM. Regular TM was used in the control wells. Cells were incubated at 17° C. for 7 d, after which the TCID_{50/mL} was determined using the Karber method (2).

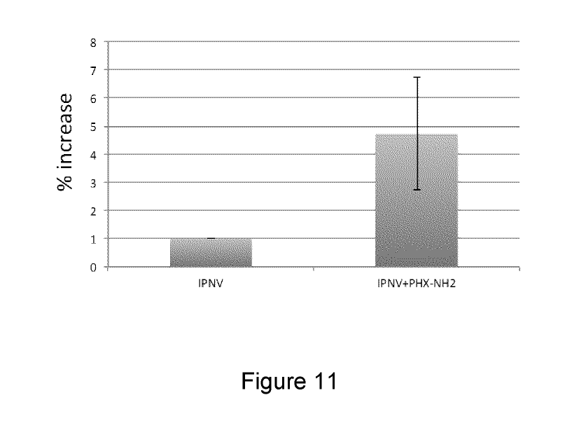

The results are shown in FIG. 11.

### Discussion

Chum salmon reovirus (CSV) and infectious pancreatic necrosis virus (IPNV) have dsRNA genomes, while viral hemorraghic septicemia virus (VHSV)-IVb is a negative sense ssRNA virus. Thus PHX-NH2 is able to affect the replication of viruses with different genomes and thus different genome replication strategies. This inhibitory effect is believed to be based on PHX-NH2's ability to suppress the host cell's type I IFN response. Trends of inhibition were observed for all three viruses; while CSV demonstrated statistically significant effects of PHX-NH2. Optimizing the extent of NH2 substitution on PHX and/or experimental conditions can provide improved enhancements.

For example, PHX-NH2 could be added to the cell culture media 2 h prior to virus infection. PHX-NH2 itself has a positive effect on cell viability, supporting cell metabolism and cell division. The virus titres following PHX-NH2 treatment would be higher than virus alone, increasing virus production rates in cell culture. This treatment would be important for cell line based virus production where type I IFNs are a confounding factor is achieving high virus yields.

### REFERENCES

(1) Reed, L. J.; Muench, H. (1938). “A simple method of estimating fifty percent endpoints”. The American Journal of Hygiene. 27: 493-497.
(2) Kärber G. (1931) Beitrag zur kollektiven Behandlung pharmakologischer Reihenversuche. Archiv f experiment Pathol u Pharmakol. 162: 480-483.

1-20. (canceled)

21. A method of suppressing an anti-viral response in a cell, a cell culture, a tissue, or a subject comprising introducing or administering an effective amount of glycogen nanoparticles or phytoglycogen nanoparticles to the cell, the cell culture, the tissue, or the subject.

22. The method of claim 21, wherein the glycogen or phytoglycogen nanoparticles are cationized.

23. The method of claim 21, wherein the glycogen or phytoglycogen nanoparticles are amine-modified.

24. The method of claim 21, wherein the glycogen or phytoglycogen nanoparticles are modified with a short-chain quaternary ammonium compound comprising at least one alkyl moiety having from 1 to 16 carbon atoms, unsubstituted or substituted with one or more N, O, S, or halogen atoms.

25. (canceled)

26. The method of claim 21, wherein at least 90% or substantially all the nanoparticles have an average diameter of between about 40 nm and about 140 nm, about 50 nm and about 130 nm, about 60 nm and about 120 nm, about 70 nm and about 110 nm, about 80 nm and about 100 nm, about 30 nm and about 40 nm, about 40 nm and about 50 nm, about 50 nm and about 60 nm, about 60 nm and about 70 nm, about 70 nm and about 80 nm, about 80 nm and about 90 nm, about 90 nm and about 100 nm, about 100 nm and about 110 nm, about 110 nm and about 120 nm, about 120 nm and about 130 nm, about 130 nm and about 140 nm, or about 140 nm and about 150 nm.

27. The method of claim 21 wherein the nanoparticles are not further conjugated to another molecule.

28. The method of claim 21, wherein the nanoparticles are further conjugated to one or more small molecules, wherein the molecule is a hydrophilicity modifier, pharmokinetic modifier, a biologically active modifier or a detectable modifier.

29. The method of claim 21, comprising administering the glycogen or phytoglycogen nanoparticles to the subject for the treatment or prevention of a disease or condition.

30. The method of claim 21, comprising administering the glycogen or phytoglycogen nanoparticles to a subject wherein the subject is a viral-vector based gene therapy patient.

31. (canceled)

32. The method of claim 29 wherein the disease or condition is an autoimmune disease or inflammatory disease.

33. (canceled)

34. The method of claim 29, wherein the disease or condition is pain.

35. The method of claim 29, wherein the disease or condition is sepsis.

36. The method of claim 29, wherein the glycogen or phytoglycogen nanoparticles are topically administered.

37. The method of claim 29, wherein the glycogen or phytoglycogen nanoparticles are systemically administered.

38. The method of claim 21 for suppressing an anti-viral response in the cell culture.

39. (canceled)

40. The method of claim 38 for enhancing viral growth and replication in an infected cell culture in the manufacture of vaccines.

41. A combination therapy comprising a viral-vector based gene therapy and glycogen or phytoglycogen nanoparticles.

42. The combination therapy of claim 41, wherein the glycogen or phytoglycogen nanoparticles are cationized.

43. (canceled)

44. The combination therapy of claim 41, wherein the glycogen or phytoglycogen nanoparticles are modified with a short-chain quaternary ammonium compound comprising at least one alkyl moiety having from 1 to 16 carbon atoms, unsubstituted or substituted with one or more N, O, S, or halogen atoms.

45. The combination therapy of claim 41, wherein the viral-vector based gene therapy is an adenovirus.
